(19) United States
(12) Patent Application Publication
(10) Pub. No.: US 2018/0322954 A1
(43) Pub. Date: Nov. 8, 2018

(54) METHOD AND DEVICE FOR CONSTRUCTING MEDICAL KNOWLEDGE GRAPH AND ASSISTANT DIAGNOSIS METHOD

(71) Applicant: Hefei University of Technology, Hefei (CN)

(72) Inventors: Shuai DING, Hefei (CN); Shanlin YANG, Hefei (CN); Xiao SUN, Hefei (CN); Shikang HU, Hefei (CN); Hao WANG, Hefei (CN)

(21) Appl. No.: 15/972,121

(22) Filed: May 5, 2018

(30) Foreign Application Priority Data
May 8, 2017 (CN) 201710317878.1

(51) Int. Cl. G16H 50/20; G16H 10/60

(52) U.S. Cl. CPC G16H 50/20; G16H 50/70; G16H 10/60

(57) ABSTRACT

The present invention discloses a method and a device for constructing a medical knowledge graph and an assistant diagnosis method. The assistant diagnosis method based on a medical knowledge graph comprises the steps of: acquiring complaint data and examination data of a patient and processing the data to obtain symptom entities and sign entities of the patient; searching, from the medical knowledge graph, disease entities associated with the symptom entities and the sign entities, calculating a posterior probability of each disease entity separately under a set of its corresponding symptom entities and sign entities; and outputting a disease entity with a maximum posterior probability as well as data corresponding to its associated nodes. The present invention provides an intelligent assistant diagnosis for clinical medicine to reduce the work burden of medical staff, relieve the medical pressure and reduce medical accidents.

### TECHNICAL FIELD

The present invention relates to the technical field of data processing, and in particular to a method and a device for constructing a medical knowledge graph and an assistant diagnosis method.

### BACKGROUND OF THE PRESENT INVENTION

As a structured information network, a knowledge graph has broken the restriction on an original relational database and possesses very strong expression ability. The knowledge graph plays an increasingly important role in fields such as information retrieval and information integration and can provide users with a broader and deeper knowledge system which expands continuously.

At present, the knowledge graph is very widely used in the field of medicine. By constructing a medical knowledge graph, a complex relationship among the symptoms, the diseases and the diagnosis and treatment measures can be constructed as a database through the knowledge graph so as to provide medical staff with a good assistant diagnosis means. However, the structure of the existing medical knowledge graphs is relatively simple, and the knowledge graph applied to assistant diagnosis cannot assist the medical staff very well due to its structural constraints.

### SUMMARY OF THE PRESENT INVENTION

Based on the above problems, the present invention provides a method and a device for constructing a medical knowledge graph and an assistant diagnosis method, which can provide intelligent assistant diagnosis for clinical medicine.

To solve the above problems, the present invention provides a method for constructing a medical knowledge graph, including the steps of:

collecting data from a medical database to construct a user dictionary;

processing electronic medical record data according to the user dictionary and a stop words library;

carrying out named entity recognition on the processed data;

establishing an association relationship among the recognized entities; and

constructing a medical knowledge graph based on the entities and the association relationship thereof.

Carrying out named entity recognition on the processed data further includes specific steps of:

obtaining disease entities from processed diagnosis data, obtaining sign entities from processed health examination data, obtaining symptom entities according to processed complaint data of a patient, obtaining treatment entities according to treatment suggestion data, and obtaining department entities according to department information.

Wherein establishing an association relationship among the recognized entities includes a specific step of:

associating the disease entities with the symptom entities, the sign entities, the treatment entities and the department entities respectively, wherein the strength of the association relationship is expressed by:

Z=x/y

where y represents the number of medical records of a certain disease, x represents the total number of occurrences of a target entity in the medical records of a certain disease, and the target entity is any one of the symptom entity, the sign entity, the treatment entity and the department entity.

Constructing a medical knowledge graph based on the entities and the association relationship thereof includes the specific steps of:

importing, into a Neo4j graphic database, entity pairs formed of the processed entities according to the association relationship, and corresponding relationship strength values thereof, and visualizing to generate the medical knowledge graph.

The method further includes a step of:

updating the strength of the association relationship between the corresponding entities in real time, based on currently obtained diagnosis results as well as the complaint data and the examination data of the patient. In another aspect, the present invention provides a computer assisted diagnosis method based on a medical knowledge graph, including the steps of:

acquiring complaint data and examination data of a patient;

preprocessing the complaint data and the examination data to obtain a set of symptom entities and sign entities of the patient;

searching, from the medical knowledge graph, a set of disease entities associated with the symptom entities and the sign entities;

calculating, based on the set of the disease entities and the set of the symptom entities and the sign entities corresponding to each disease entity, a posterior probability of each disease entity separately under a set of its corresponding symptom entities and sign entities; and

outputting a disease entity with a maximum posterior probability as well as data corresponding to its associated nodes.

Calculating a posterior probability of each disease entity under its corresponding subset includes a specific step of:

calculating a posterior probability of a disease dᵢ under a set {t₁, t₂, . . . tₖ} of the corresponding symptom entities and sign entities:

$$P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} )}{P ( {t}_{1} , {t}_{2} , … {t}_{k} )} ,$$

Where

P(t₁, t₂ . . . tₖ)=Σ₁ⁿP(t₁|dᵢ)P(t₂|dᵢ) . . . P(tₖ|dᵢ), n is the number of disease entities, and k is the number of symptom entities and sign entities.

through the relationship strength value between the symptom entities and the disease entities in the knowledge graph, a posterior probability of disease entities is calculated under the symptom entities, and a disease d with the largest posterior probability as well as data of the associated node is returned.

In still another aspect, the present invention provides a device for constructing a medical knowledge graph, including:

a user dictionary construction unit configured to collect data from a medical database to construct a user dictionary;

a data processing unit configured to process electronic medical record data according to the user dictionary and a stop words library;

an entity recognition unit configured to carry out named entity recognition on the data processed by the data processing unit; and

an association relationship establishment unit configured to establish an association relationship among the entities formed by the entity recognition unit; and

a medical knowledge graph construction unit configured to construct a medical knowledge graph based on the entities and the association relationship thereof.

The entity recognition unit specifically includes:

a disease entity recognition subunit configured to carry out named entity recognition on the processed diagnosis data to obtain disease entities;

a sign entity recognition subunit configured to carry out named entity recognition on the processed health examination data to obtain sign entities;

a symptom entity recognition subunit configured to carry out named entity recognition on the processed complaint data to obtain symptom entities;

a treatment entity recognition subunit configured to carry out named entity recognition on the processed treatment suggestion data to obtain treatment entities; and

a department entity recognition subunit configured to carry out named entity recognition on the processed department information to obtain department entities.

The device further includes:

an updating unit configured to update the strength of the association relationship between the corresponding entities in real time, based on currently obtained diagnosis results as well as the complaint data and examination data of the patient. By the method and device for constructing a medical knowledge graph and the assistant diagnosis method provided by the present invention, the diagnosis can be assisted by the medical knowledge graph to reduce the work burden for medical staff and effectively relieve the medical pressure, thereby reducing medical accidents. At the same time, an accurate computer assisted diagnosis, which provides strong support for the medical staff, is provided for cases that cannot be diagnosed by experience of the medical staff.

In addition, persons of ordinary without relevant medical knowledge background, through their own symptoms, can understand basic and feasible methods against diseases based on information and corresponding treatment suggestions about the diseases they suffer in the system.

### BRIEF DESCRIPTION OF THE DRAWINGS

FIG. 1 shows a flowchart of a method for constructing a medical knowledge graph of the present invention;

FIG. 2 shows a flowchart of an assistant diagnosis method based on a medical knowledge graph of the present invention; and

FIG. 3 shows a device for constructing a medical knowledge graph of the present invention.

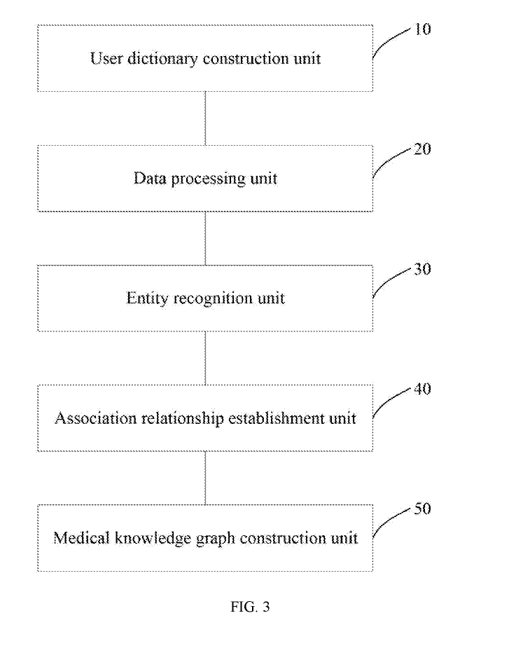

### DETAILED DESCRIPTION OF THE PRESENT INVENTION

The specific embodiments of the present invention will be further described in detail with reference to the drawings by the embodiments. The following embodiments are used for describing the present invention, but not for limiting the scope thereof.

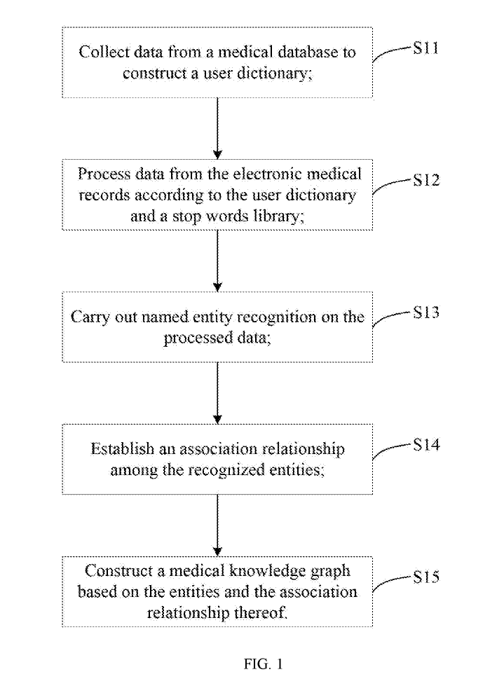

FIG. 1 shows a flowchart of a method for constructing a medical knowledge graph of the present invention.

With reference to FIG. 1, the method for constructing a medical knowledge graph provided by the present invention specifically includes:

S11. Data is collected from a medical database to construct a user dictionary.

In this embodiment, relevant data are collected according to ICD-10 and ICD-9-CM in an existing medical database to construct a user dictionary.

S12. Data from the electronic medical records is processed according to the user dictionary and a stop words library.

In this embodiment, the data used for constructing the knowledge graph may be data obtained from the existing electronic medical records, such as patient's complaints, department, medical history, health examination, diagnosis, treatment suggestions and other related data. The data from the electronic medical records can be processed by a related medical language processing technology (MLP) according to the user dictionary and the stop words library, and can also be processed by word segmentation and stop words elimination, enabling the data used for constructing the knowledge graph to be more accurate.

S13. Named entity recognition is carried out on the processed data.

In one embodiment, a CRF++ tool may be used for carrying out named entity recognition by a Conditional Random Fields Model (CRF) machine learning method. For example, in one embodiment, disease entities are obtained from processed diagnosis data, sign entities are obtained from processed health examination data, symptom entities are obtained according to processed complaint data of a patient, treatment entities are obtained according to treatment suggestion data, and department entities are obtained according to department information.

S14. An association relationship is established among the recognized entities.

In the embodiment, an association relationship is established between the disease entities and the symptom entities, the sign entities, the treatment entities and the department entities respectively, and the strength of the association relationship is expressed by:

Z=x/y

where y represents the number of medical records of a certain disease, x represents the total number of occurrences of a target entity in the medical records of a certain disease, and the target entity is any one of the symptom entity, the sign entity, the treatment entity and the department entity.

S15. A medical knowledge graph is constructed based on the entities and the association relationship thereof.

Specifically, it includes steps of importing, into a Neo4j graphic database, entity pairs formed of the processed entities according to the association relationship, and corresponding relationship strength values thereof, and visualizing to generate a medical knowledge graph.

In a further embodiment, after the knowledge graph is used for assisting the diagnosis, the strength of the association relationship between the corresponding entities in real time is updated based on the diagnosis results of the disease as well as the complaint data and examination data of the patient, and its relationship strength is Z=(x+1)/(y+1), where y is the number of processed and recorded medical records of the disease and x is the number of occurrences of the target entity in the processed and recorded medical records of the disease.

In the medical knowledge graph of the present invention, the electronic medical records are used as source data for constructing the knowledge graph, and an accurate knowledge graph can be constructed by establishing an association relationship among the entities so as to assist the medical staff against diseases well. It can reduce the work burden for medical staff and effectively relieve the medical pressure, thereby reducing medical accidents.

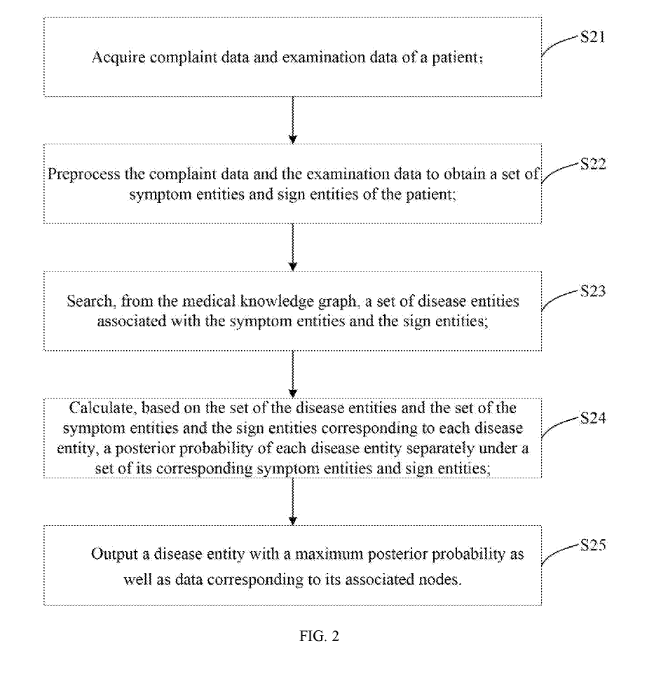

In another embodiment, the present invention provides an assistant diagnosis method based on a medical knowledge graph. As shown in FIG. 2, the method specifically includes:

S21. Complaint data and examination data of a patient are acquired.

S22. The complaint data and the examination data are preprocessed to obtain a set of symptom entities and sign entities of the patient. For example, the complaint data and the examination data are processed by the word segmentation, stop words elimination and named entity recognition to obtain a set of the symptom entities and the sign entities.

S23. A set of disease entities associated with the symptom entities and the sign entities is searched from the medical knowledge graph.

A set of the disease entities D{d₁, d₂, . . . dₙ} is searched from the medical knowledge graph according to the association relationship between the entities in the set of the symptom entities and the sign entities obtained in step S22 and the disease entities.

In the above process, a conditional probability P(sⱼ|dᵢ) of the disease dᵢ to a symptom entity or a sign entity sⱼ can be set as a relationship strength value between two entities, i.e., P(sⱼ|dᵢ)=x/y.

S24. Based on the disease entities obtained from S23 and the set of the symptom entities and the sign entities corresponding to each disease entity obtained from S222, a posterior probability of each disease entity is calculated separately under a set of its corresponding symptom entities and sign entities.

S25. A disease entity with a maximum posterior probability and data corresponding to its associated nodes are output.

In the above process, corresponding to n disease entities in the set D of the disease entities, n sets corresponding to the symptom entities and the sign entities are found through the association relationship. A posterior probability of a disease dᵢ under a set {t₁, t₂, . . . tₖ} of the corresponding symptom entities and sign entities is calculated:

$$P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} )}{P ( {t}_{1} , {t}_{2} , … {t}_{k} )} ,$$

where P(t₁, t₂ . . . tₖ)=Σ₁ⁿP(t₁|dᵢ)P(t₂|dᵢ) . . . P(tₖ|dᵢ), n is the number of disease entities, and k is the number of symptom entities and sign entities, i.e., the posterior probability is:

$$\begin{matrix}P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} ) P ( {d}_{i} )}{{\sum }_{1}^{n} P ( {t}_{1} \vert {d}_{i} ) P ( {t}_{2} \vert {d}_{i} ) … P ( {t}_{k} \vert {d}_{i} ) P ( {d}_{i} )} \\ = \frac{P ( {t}_{1} \vert {d}_{i} ) P ( {t}_{2} \vert {d}_{i} ) … P ( {t}_{k} \vert {d}_{i} )}{{\sum }_{i}^{n} P ( {t}_{1} \vert {d}_{i} ) P ( {t}_{2} \vert {d}_{i} ) … P ( {t}_{k} \vert {d}_{i} )}\end{matrix}$$

By the calculated posterior probabilities, a disease with a maximum posterior probability and data corresponding to its associated nodes are used as diagnosis results.

In this embodiment, the computer assisted diagnosis method based on a medical knowledge graph includes the steps of collecting complaint data and examination data of a patient by using a terminal device, carrying out medical language processing MLP (word segmentation and elimination of stop words) and named entity recognition on the data to obtain information about corresponding entities, finding a corresponding set of candidate diseases through the association relationship based on the medical knowledge graph that has been constructed, and then assisting the diagnosis by the Bayesian algorithm to confirm the type of disease and provide an intelligent assistant diagnosis for clinical medicine.

In still another embodiment, the present invention provides a device for constructing a medical knowledge graph. As shown in FIG. 2, the device includes:

a user dictionary construction unit 10 configured to collect data from a medical database to construct a user dictionary;

a data processing unit 20 configured to process electronic medical record data according to the user dictionary and a stop words library;

an entity recognition unit 30 configured to carry out named entity recognition on the data processed by the data processing unit; and

an association relationship establishment unit 40 configured to establish an association relationship among the entities formed by the entity recognition unit; and

a medical knowledge graph construction unit 50 configured to construct a medical knowledge graph based on the entities and the association relationship thereof.

Specifically, in the above embodiment, the entity recognition unit 30 includes:

a disease entity recognition subunit configured to carry out named entity recognition on the processed diagnosis data to obtain disease entities;

a sign entity recognition subunit configured to carry out named entity recognition on the processed health examination data to obtain sign entities;

a symptom entity recognition subunit configured to carry out named entity recognition on the processed complaint data to obtain symptom entities;

a treatment entity recognition subunit configured to carry out named entity recognition on the processed treatment suggestion data to obtain treatment entities; and

a department entity recognition subunit configured to carry out named entity recognition on the processed department information to obtain department entities.

In still another embodiment, the present invention provides a device for constructing a medical knowledge graph, including an updating unit configured to update the strength of the association relationship between the corresponding entities in real time, based on currently obtained diagnosis results as well as the complaint data and examination data of the patient.

By the method and device for constructing a medical knowledge graph and the assistant diagnosis method provided by the present invention, the diagnosis can be can assisted by the medical knowledge graph to reduce the work burden for medical staff and effectively relieve the medical pressure, thereby reducing medical accidents. At the same time, an accurate computer assisted diagnosis, which provides strong support for the medical staff, is provided for cases that cannot be diagnosed by experience of the medical staff.

In addition, persons of ordinary without relevant medical knowledge background, through their own symptoms, can understand basic and feasible methods against diseases based on information and corresponding treatment suggestions about the diseases they suffer in the system.

The above embodiments are merely used for describing the present invention, but not for limiting the present invention. A person of ordinary skill in the art may also make various changes and modifications without departing from the spirit and scope of the present invention. Therefore, all equivalent technical solutions also fall into the scope of the present invention, and the patent protection scope of the present invention should be defined by the claims.

What is claimed is:

1. A method and device for establishing medical knowledge graph, comprising the steps of:
collecting data from a medical database to construct a user dictionary;
processing electronic medical record data according to the user dictionary and a stop words library;
carrying out named entity recognition by a Conditional Random Fields Model (CRF) machine learning method on the processed data;
establishing an association relationship among the recognized entities; and
establishing a medical knowledge graph based on the entities and the association relationship thereof;
using relevant medical language processing techniques to process the data of the electronic medical records, or perform text segmentation on the data of the electronic medical records, and remove stop word processing;
obtaining disease entities from processed diagnosis data, obtaining sign entities from processed health examination data, obtaining symptom entities according to processed complaint data of a patient, obtaining treatment entities according to treatment suggestion data, and obtaining department entities according to department information;
associating the disease entities with the symptom entities, the sign entities, the treatment entities and the department entities respectively, wherein the strength of the association relationship is expressed by:
Z=x/y
where y represents the number of medical records of a certain disease, x represents the total number of occurrences of a target entity in the medical records of a certain disease, and the target entity is any one of the symptom entity, the sign entity, the treatment entity and the department entity.

2. The method according to claim 1, wherein constructing a medical knowledge graph based on the entities and the association relationship thereof comprises the specific steps of:
importing, into a Neo4j graphic database, entity pairs formed of the processed entities according to the association relationship, and corresponding relationship strength values thereof, and visualizing to generate the medical knowledge graph.

3. The method according to claim 1, wherein the method further comprises a step of:
updating the strength of the association relationship between the corresponding entities in real time, based on currently obtained diagnosis results as well as the complaint data and the examination data of the patient.

4. An auxiliary query method for a medical knowledge graph based on the method according to claim 1, comprising the steps of:
acquiring complaint data and examination data of a patient;
preprocessing the complaint data and the examination data to obtain a set of symptom entities and sign entities of the patient;
searching, from the medical knowledge graph, a set of disease entities associated with the symptom entities and the sign entities;
calculating, based on the set of the disease entities and the set of the symptom entities and the sign entities corresponding to each disease entity, a posterior probability of each disease entity separately under a set of its corresponding symptom entities and sign entities; and
outputting a disease entity with a maximum posterior probability as well as data corresponding to its associated nodes.

5. An auxiliary query method for a medical knowledge graph based on the method according to claim 2, comprising the steps of:
acquiring complaint data and examination data of a patient;
preprocessing the complaint data and the examination data to obtain a set of symptom entities and sign entities of the patient;
searching, from the medical knowledge graph, a set of disease entities associated with the symptom entities and the sign entities;
calculating, based on the set of the disease entities and the set of the symptom entities and the sign entities corresponding to each disease entity, a posterior probability of each disease entity separately under a set of its corresponding symptom entities and sign entities; and
outputting a disease entity with a maximum posterior probability as well as data corresponding to its associated nodes.

6. An auxiliary query method for a medical knowledge graph based on the method according to claim 3, comprising the steps of:
acquiring complaint data and examination data of a patient;
preprocessing the complaint data and the examination data to obtain a set of symptom entities and sign entities of the patient;
searching, from the medical knowledge graph, a set of disease entities associated with the symptom entities and the sign entities;
calculating, based on the set of the disease entities and the set of the symptom entities and the sign entities corresponding to each disease entity, a posterior probability of each disease entity separately under a set of its corresponding symptom entities and sign entities; and
outputting a disease entity with a maximum posterior probability as well as data corresponding to its associated nodes.

7. The auxiliary query method according to claim 4, wherein calculating a posterior probability of each disease entity under its corresponding subset comprises a specific step of:
calculating a posterior probability of a disease dᵢ under a set {t₁, t₂, . . . tk} of the corresponding symptom entities and sign entities:
$$P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} )}{P ( {t}_{1} , {t}_{2} … {t}_{k} )} , \\ where$$
$$P ( {t}_{1} , {t}_{2} , … {t}_{k} ) = {\sum }_{1}^{n} P ( {t}_{1} \vert {d}_{i} ) P ( {t}_{2} \vert {d}_{i} ) … P ( {t}_{k} \vert {d}_{i} ) ,$$
n is the number of disease entities, and k is the number of symptom entities and sign entities.

8. The auxiliary query method according to claim 5, wherein calculating a posterior probability of each disease entity under its corresponding subset comprises a specific step of:
calculating a posterior probability of a disease dᵢ under a set {t₁, t₂, . . . tk} of the corresponding symptom entities and sign entities:
$$P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} )}{P ( {t}_{1} , {t}_{2} … {t}_{k} )} , \\ where$$
$$P ( {t}_{1} , {t}_{2} , … {t}_{k} ) = {\sum }_{1}^{n} P ( {t}_{1} \vert {d}_{i} ) P ( {t}_{2} \vert {d}_{i} ) … P ( {t}_{k} \vert {d}_{i} ) ,$$
n is the number of disease entities, and k is the number of symptom entities and sign entities.

9. The auxiliary query method according to claim 6, wherein calculating a posterior probability of each disease entity under its corresponding subset comprises a specific step of:
calculating a posterior probability of a disease dᵢ under a set {t₁, t₂, . . . tk} of the corresponding symptom entities and sign entities:
$$P ( {d}_{i} \vert {t}_{1} , {t}_{2} , … {t}_{k} ) = \frac{P ( {t}_{1} \vert {d}_{i} ) P ( {t}_{2} \vert {d}_{i} ) … P ( {t}_{k} \vert {d}_{i} )}{P ( {t}_{1} , {t}_{2} … {t}_{k} )} , \\ where$$
$$P ( {t}_{1} , {t}_{2} , … {t}_{k} ) = {\sum }_{1}^{n} P ( {t}_{1} \vert {d}_{i} ) P ( {t}_{2} \vert {d}_{i} ) … P ( {t}_{k} \vert {d}_{i} ) ,$$
n is the number of disease entities, and k is the number of symptom entities and sign entities.

10. A device for establishing a medical knowledge graph, comprising:
a user dictionary construction unit configured to collect data from a medical database to construct a user dictionary;
a data processing unit configured to process electronic medical record data according to the user dictionary and a stop words library;
an entity recognition unit configured to carry out named entity recognition on the data processed by the data processing unit; and
an association relationship establishment unit configured to establish an association relationship among the entities formed by the entity recognition unit; and
a medical knowledge graph construction unit configured to construct a medical knowledge graph based on the entities and the association relationship thereof;
the entity recognition unit specifically comprises:
a disease entity recognition subunit configured to carry out named entity recognition on the processed diagnosis data to obtain disease entities;
a sign entity recognition subunit configured to carry out named entity recognition on the processed health examination data to obtain sign entities;
a symptom entity recognition subunit configured to carry out named entity recognition on the processed complaint data to obtain symptom entities;
a treatment entity recognition subunit configured to carry out named entity recognition on the processed treatment suggestion data to obtain treatment entities; and
a department entity recognition subunit configured to carry out named entity recognition on the processed department information to obtain department entities;
an updating unit configured to update the strength of the association relationship between the corresponding entities in real time, based on currently obtained diagnosis results as well as the complaint data and examination data of the patient.
